(19) United States
(12) Patent Application Publication
(10) Pub. No.: US 2026/0132457 A1
(43) Pub. Date: May 14, 2026

(54) SEQUENCING SCAFFOLD AND METHOD

(71) Applicant: Leica Microsystems CMS GmbH, Wetzlar (DE)

(72) Inventor: Soeren ALSHEIMER, Wetzlar (DE)

(21) Appl. No.: 19/105,048

(22) PCT Filed: Nov. 18, 2022
(86) PCT No.: PCT/EP2022/082454

§ 371 Date: Feb. 20, 2025

(30) Foreign Application Priority Data
Aug. 23, 2022 (EP) 22191689.3
Aug. 23, 2022 (EP) 22191690.1

(51) Int. Cl. C12Q 1/6874

(52) U.S. Cl. CPC C12Q 1/6874

(57) ABSTRACT

A sequencing scaffold for attachment of target nucleic acid strands includes at least one nucleic acid backbone with a plurality of attachment regions at predetermined positions. Each attachment region includes at least one attachment site. The attachment sites are configured to hybridise the target nucleic acid strands. The nucleic acid backbone has a three-dimensional lattice structure. The attachment regions are arranged on the lattice structure of the nucleic acid backbone in three dimensions.

### CROSS REFERENCE TO RELATED APPLICATIONS

This application is a U.S. National Phase application under 35 U.S.C. § 371 of International Application No. PCT/EP2022/082454, filed on Nov. 18, 2022, and claims benefit to European Patent Application No. EP 22191690.1, filed on Aug. 23, 2022 and European Patent Application No. EP 22191689.3, filed on Aug. 23, 2022. The International Application was published in English on Feb. 29, 2024 as WO 2024/041749 A1 under PCT Article 21(2).

### FIELD

Embodiments of the invention relate to a sequencing scaffold for attachment of target nucleic acid strands and a method for sequencing the target nucleic acid strands.

### BACKGROUND

Nucleic acid sequencing has continuously improved from early methods such as Maxam-Gilbert or Sanger sequencing to massively parallel sequencing such as 454 sequencing, Illumina sequencing or DNA Nanoball sequencing. While earlier sequencing methods allowed for higher read lengths, they were severely limited in terms of throughput. In contrast, massively parallel sequencing methods use miniaturised and parallelised approaches for sequencing a very large number of short reads that are subsequently assembled into longer sequences. However, there is a constant drive to further improve these methods to provide higher throughput methods, enabling still faster sequencing results.

### SUMMARY

Embodiments of the present invention provide a sequencing scaffold for attachment of target nucleic acid strands. The sequencing scaffold includes at least one nucleic acid backbone with a plurality of attachment regions at predetermined positions. Each attachment region includes at least one attachment site. The attachment sites are configured to hybridise the target nucleic acid strands. The nucleic acid backbone has a three-dimensional lattice structure. The attachment regions are arranged on the lattice structure of the nucleic acid backbone in three dimensions.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view of several nucleic acid backbones with orientation indicators and attachment regions according to some embodiments;

FIG. 2A is a first schematic view of a nucleic acid backbone of a sequencing scaffold with attached target nucleic acids strand according to some embodiments;

FIG. 2B is a first schematic view of a nucleic acid backbone of a sequencing scaffold with attached target nucleic acids strand and a docking strand according to some embodiments;

FIG. 3 is a schematic view of the nucleic acid backbone according to FIGS. 2A and 2B with sequencing information of attached target nucleic acid strands according to some embodiments;

FIG. 4 is a schematic view of the DNA nanoball sequencing on a sequencing scaffold after rolling circle amplification according to some embodiments;

FIG. 5A is schematic view of steps S500 to S504 of a sequencing method for a sequencing scaffold according to some embodiments;

FIG. 5B is schematic view of a step S506 of the sequencing method according to some embodiments;

FIG. 5C is schematic view of a step S508 of the sequencing method according to some embodiments;

FIG. 5D is schematic view of a step S510 of the sequencing method according to some embodiments;

FIG. 5E is schematic view of a step S512 of the sequencing method according to some embodiments;

FIG. 5F is schematic view of a step S514 of the sequencing method according to some embodiments;

FIG. 5G is schematic view of a step S516 of the sequencing method according to some embodiments;

FIG. 5H is schematic view of a step S518 of the sequencing method according to some embodiments;

FIG. 5I is schematic view of a step S520 of the sequencing method according to some embodiments; and

FIG. 5J is schematic view of a step S522 of the sequencing method according to some embodiments.

### DETAILED DESCRIPTION

Embodiments of the present invention provide a device and method that allow improved high-throughput sequencing.

A sequencing scaffold for attachment of target nucleic acid strands comprises at least one nucleic acid backbone with a plurality of attachment regions at predetermined positions, each attachment region comprising at least one attachment site, and the nucleic acid backbone having a three-dimensional lattice structure and the attachment regions being arranged on the lattice structure of the nucleic acid backbone in three dimensions.

Thus, the nucleic acid backbone provides anchor points for the target nucleic acids, meaning for nucleic acid strands to be sequenced, for example, by pyrosequencing method. The lattice structure of the backbone extends in three dimensions and the attachment regions are distributed over the backbone in three dimensions. This is to be understood in contrast to a surface for example of a microarray, a flow cell, or the walls of a microfluidic channel, which only provide two-dimensional surfaces, to which target nucleic acid strands may be attached. Thus, by providing a three-dimensional lattice structure, the number of attachment regions in a given space may be increased substantially compared to a two-dimensional attachment surface.

Preferably, the sequencing scaffold comprises at least first, second and third orientation indicators. These may be fluorescent dyes, for example, and enable determining the orientation of the scaffold in space.

It is particularly preferred, when the orientation indicators are spaced apart from each other, or arranged on opposite ends of the backbone. For example, the orientation indicators, in particular the respective fluorescent dyes, may further differ from each other in an optical property such as excitation wavelength, emission wavelength or emission lifetime. Thus, the orientation indicators are attached to the backbone and can be distinguished from each other. Thus, the orientation indicators enable identifying respective ends of the backbone and therefore its orientation in space.

Preferably, the attachment regions are arranged on the backbone in several, in particular parallel, planes and the planes are arranged at a distance from each other in a direction perpendicular to the planes. This enables regularly spaced attachment regions and a high density of attachment regions in a given volume.

Preferably, the distance between the planes is in a range from 5 nm to 6400 nm, preferably in a range from 10 nm to 3200 nm, more preferably in a range from 50 nm to 800 nm. This enables a particularly high density of attachment regions in a given volume. The spacing between the planes may be chosen depending on the resolving power of a readout device, e.g. of a microscope, used to read out sequencing reactions when sequencing the target nucleic acids.

Preferably, the largest spatial extent of the backbone is in a range of 10 nm to 10000 nm, preferably in a range from 0.1 μm to 5 μm. This enables a particularly compact scaffold, that may be integrated in flow devices for sequencing, such as microfluidic chips.

Preferably, the sequencing scaffold is attached to a wall of a flow device or a microfluidic chip, wherein the wall is adapted to enable an optical readout, e.g. by a microscope. The wall might, for example be optically transparent.

Preferably, the attachment regions are spaced apart from each other in a range from 1 nm to 5000 nm, preferably in a range from 5 nm to 1000 nm, more preferably in a range from 10 nm to 500 nm. This enables a particularly dense arrangement of the target nucleic acids on the backbone.

Preferably, for each attachment region, the attachment sites are spaced apart in a range from 1 nm to 500 nm, preferably in a range from 10 nm to 200 nm. The spacing between the attachment sites may be chosen depending on the resolving power of a readout device used to read out sequencing reactions when sequencing the target nucleic acids. Particularly preferable ranges may correspond to the lateral resolution achievable with different microscopic modalities such as, MINFLUX (<1 nm to 10 nm), single molecule localization microscopy (1 nm to 25 nm), structured illumination and/or STED microscopy (50 nm to 100 nm), high NA (numerical aperture) light microscopy (around 200 nm), and low NA light microscopy (around 500 nm). Importantly, the attachment sites may be distanced from each other such that the readout device can resolve them individually.

Similar ranges regarding the typical axial optical resolution (being lower than the typical lateral optical resolution) of the optical readout being achievable with different microscopic modalities might apply. In particular, the attachment sites are spaced apart from each other corresponding to the axial resolution achievable with different microscopic modalities. For example, the nucleic acid backbone can be generated such that the attachment sites are arranged on the backbone in several planes, wherein the distances between these planes from each other are in a direction perpendicular to the planes and are larger than the typical axial optical resolution of the optical readout. Thus, an optical readout of the sequencing reactions can be resolved in the axial direction of the optical readout system, as well.

Using microscopic readout modalities, like MINFLUX, single molecule localization microscopy, structured illumination or STED for example, very dense packing of attachment sites can be realised, with attachment sites being spaced a few nanometres apart and wherein the readout can still assign a signal from a sequencing reaction to a certain target nucleic acid attached to a certain attachment site. In this way the number of reads that can be generated from a single flow cell lane is greatly improved. A suitable spacing of attachment sites in three dimensions may also take the length of nucleic acid targets that shall be attached and sequenced into account, wherein in a DNA oligonucleotide of 300 bp length is about 100 nm long.

Preferably, the backbone comprises scaffold strands, which may be longer nucleic acid strands, for example M13 phage genome DNA, and staple strands, the staple strands configured to bind to the scaffold strands at predetermined positions to fold the scaffold strands into a predetermined shape. Thus, the backbone may be a DNA origami backbone. These DNA origami structures may range in size from a few nanometres into the micron range. For the fabrication of such DNA origami-based structures longer DNA molecules (scaffold strands) are folded at precisely identified positions by so-called staple strands. The DNA origami may be designed to provide a self-assembly backbone of a particular predetermined shape. This enables an easy and reproducible synthesis and assembly of the backbone. Staple strands may be position-selectively functionalised. The positional resolution in this case is limited by the size of a nucleotide, which is in the range of a nanometre or below. This has been exploited in the prior art to generate fluorescent standards, wherein fluorescent dyes are connected to precisely located bands on the DNA origami. These standards are known as “nanoruler” and are used for the calibration of imaging systems like confocal or super resolution microscopes (e.g. STED), for example, as disclosed by US2014/0057805 A1.

The DNA origami provides a scaffold for the target nucleic acid strands. Preferably, the DNA origami backbone comprises at least one scaffold strand and multiple staple strands, wherein the staple strands are complementary to at least parts of the scaffold strand and configured to bring the scaffold strand into a predetermined conformation. In particular, the staple strands are oligonucleotides. This enables generating nucleic acid backbones with predetermined two- or three-dimensional shapes that can self-assemble. Further, this enables the site-specific placement of attachment sites on the backbone using special staple strands that serve as attachment sites (also referred to as docking strands). In the sense of this document a staple strand partially comprising a sequence, which does not bind to the scaffold strand and serves as an attachment site or other form of functionalization may also be referred to as a docking strand.

Alternatively, the nucleic acid backbone may comprise or consist of DNA bricks, which typically comprise four domains (1-4). These DNA bricks are shorter length oligonucleotides that have overlapping hybridising stretches in order to bind to each other and assemble the backbone. In the case of DNA bricks attachment sites can be formed by extending certain DNA bricks by at least one further domain, i.e. a 5th, which serves as an attachment site and that has no hybridizing partner domain in the set of DNA bricks that is used to assemble the DNA-brick based nucleic acid backbone.

Preferably, the staple strands comprise the attachment sites in which case they may also be referred to as docking strands. The attachment sites are nucleic acid sequences, preferably of the staple strands. Preferably, the target nucleic acids may be attached to the staple strands of the backbone at predetermined attachment sites. Since the staple strands are located at predetermined positions the positions of the attachment sites may equally be predetermined. Thus, the attachment site is an oligonucleotide sequence configured to bind the target nucleic acid strand.

In a preferred embodiment, the backbone comprises double stranded oligonucleotides and the attachment sites comprise single stranded oligonucleotides. Thus, the target nucleic acids may only bind at the attachment sites and random binding to the rest of the backbone is avoided.

Preferably, each attachment sites comprises a unique oligonucleotide sequence. In particular, these attachment sites may be random. This enables a large number of different target nucleic acid strands to hybridise directly to the attachment sites.

Preferably, each attachment region comprises at least a first set of attachment sites, configured to hybridise a first adapter oligonucleotide and a second set of attachment sites, configured to hybridise a second adapter oligonucleotide. This enables particular efficient sequencing of the target strands.

More preferably, the first and second oligonucleotide adapters are configured to be ligated to the target nucleic acid strands.

Preferably, the scaffold comprises a second nucleic acid backbone, and the nucleic acid backbones are linked to each other. This link may be a nucleic acid link or a chemical, non-nucleic acid link. This enables assembling a larger scaffold with several backbones.

In another aspect a method is provided for sequencing target nucleic acid strands, comprising the following steps: Providing a scaffold comprising at least one nucleic acid backbone; attaching, directly or indirectly, the target nucleic acid strands to attachment sites of the at least one nucleic acid backbone of the scaffold; sequencing the target nucleic acid strands according to a sequencing method, the sequencing method comprising at least a step of amplifying the attached target nucleic acid strands and a step of iteratively optically reading out the attachment sites.

The sequencing method may in particular be a method that requires optically reading out sequencing reactions of the target nucleic acid strands attached to respective attachment sites. For example, a pyrosequencing method or a dye termination method such as Illumina or 454 sequencing.

Preferably, prior to the step of attaching the target nucleic acid strands to the attachment sites, the first adapter oligonucleotide and the second adapter oligonucleotide are ligated to each target nucleic acid strand.

The method has the same advantages as the sequencing scaffold described above and can be supplemented using the features of the dependent claims directed at the sequencing scaffold.

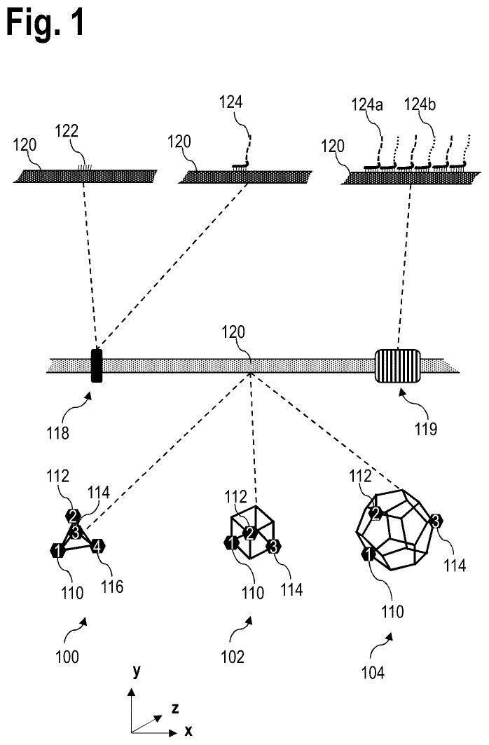

FIG. 1 is a schematic view of several three-dimensional nucleic acid backbones 100, 102, 104 of a sequencing scaffold. Generally, the nucleic acid backbones 100, 102, 104 consist of a plurality of nucleic acid strands 120. In particular, the backbones 100, 102, 104 are based on structural DNA nanotechnology (e.g. DNA tiles-, DNA origami-, DNA bricks-based structures, or other DNA nanotechnology-based structures), which allows generating predetermined, stable three-dimensional shapes.

The backbones may have a variety of geometries, for example, the tetrahedral backbone 100, the cuboidal, in particular cubic, backbone 102, or the polyhedral backbone 104. These three-dimensional geometries of the backbone 100, 102, 104 enable arranging the attachment sites on the backbones 100, 102, 104 in three dimensions. In contrast to two-dimensional structures, for example sheet-like or rod-like structures, these three-dimensional structures can provide a larger number of attachment sites for a given base area. The geometries may be arbitrary and may include solid, wireframe-like, or hollow structures.

The nucleic acid backbones 100, 102, 104 allow generating a plurality of attachment regions 118, 119 at predetermined positions along the backbones 100, 102, 104. The attachment regions 118, 119 are stretches of nucleic acids, that allow the hybridisation of complementary oligonucleotides, for example target nucleic acid strands, to the backbones 100, 102, 104 at these predetermined positions.

In particular, the backbones 100, 102, 104 may comprise the attachment regions 118 with integrated attachment sites 122 that are short sequence stretches of the nucleic acid strands 120 of the backbones 100, 102, 104. Alternatively, the attachment regions 118 of the backbones 100, 102, 104 may comprise connected attachment sites 124, that are arranged on a nucleic acid staple strand (also termed a docking strand) of the backbones 100, 102, 104 that is partially complementary to a part of the nucleic acid strand 120 of the backbone. Thus, target nucleic acid strands may hybridise either directly or indirectly to the nucleic acid strands 120 of the backbones 100, 102, 104.

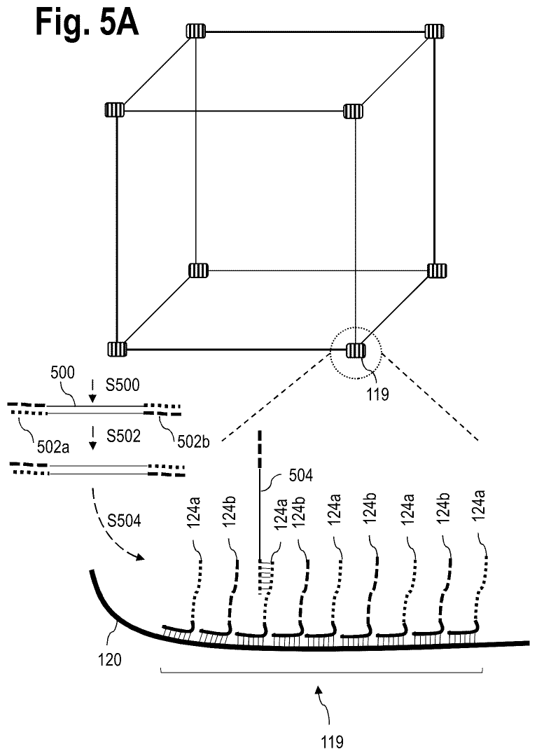

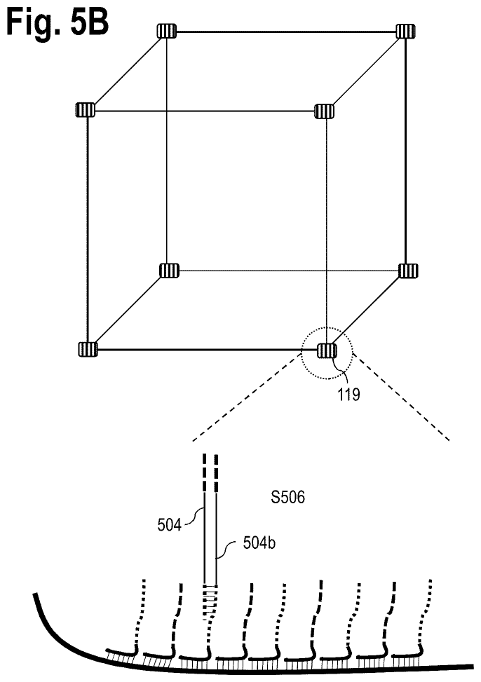

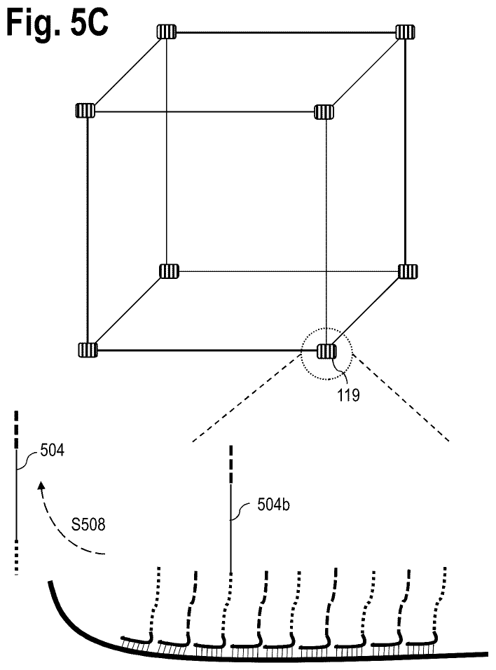

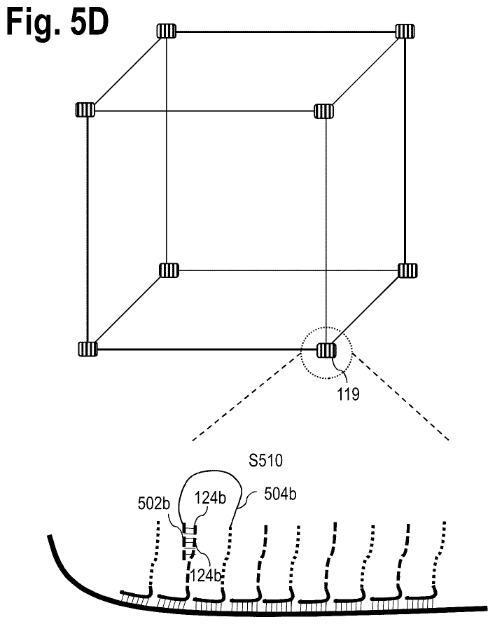

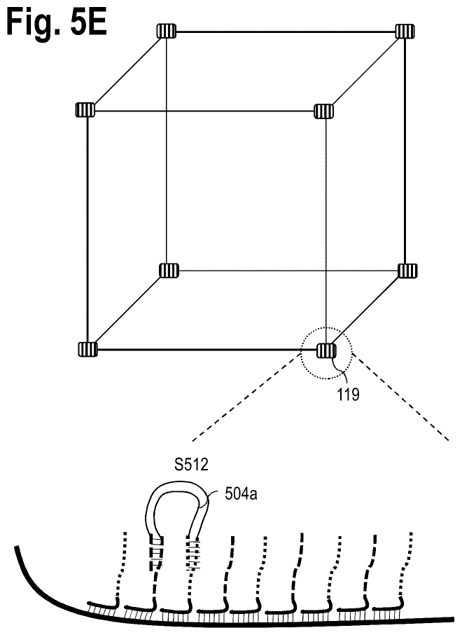

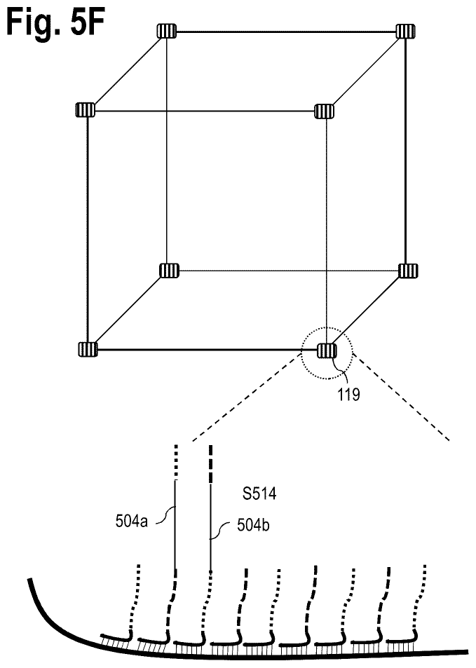

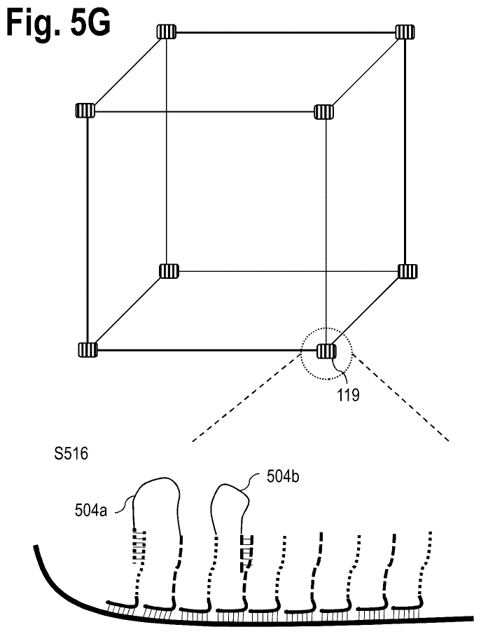

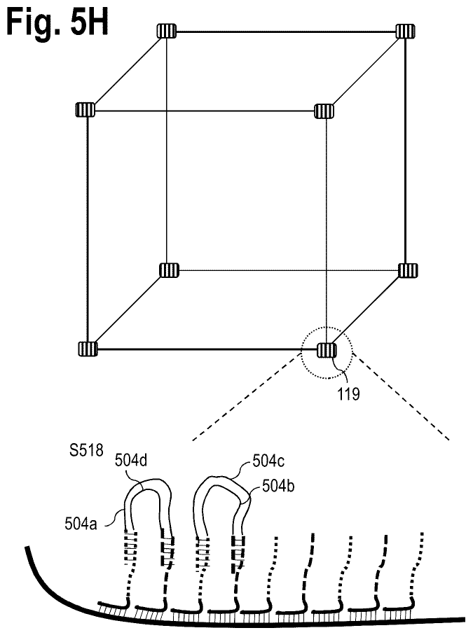

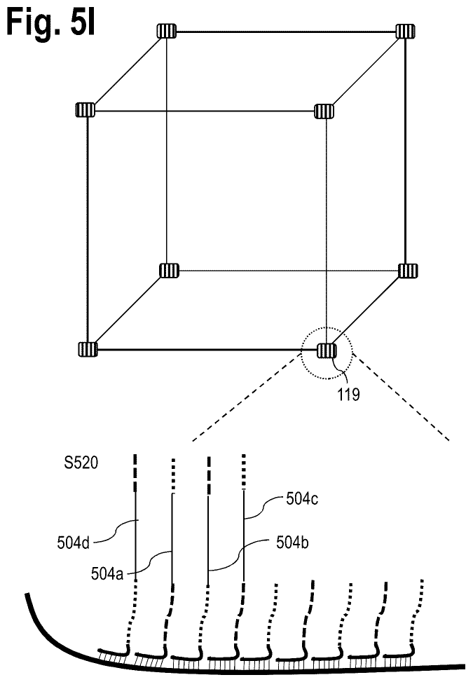

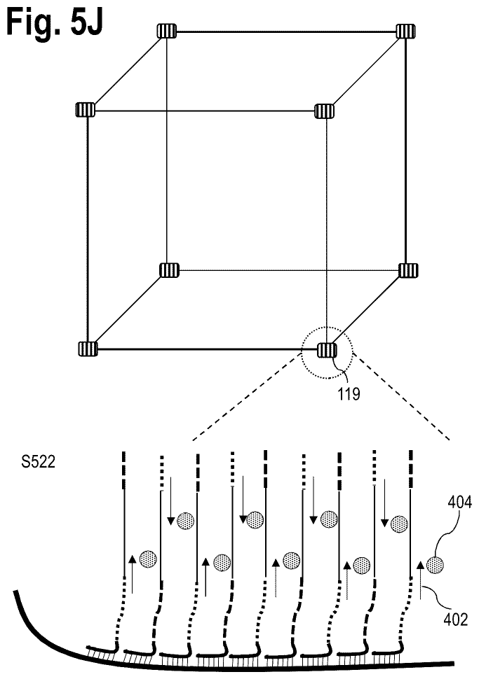

Alternatively or in addition, the backbones 100, 102, 104 may comprise the attachment regions 119. The attachment regions 119 comprise a plurality of attachment sites. Specifically, the attachment regions 119 comprise several first attachment sites 124a and several second attachment sites 124b. These attachment sites 124a, 124b support specific target nucleic acid amplification strategies and chemistries on the backbones 100, 102, 104. For example, a plurality of the attachment regions 119 is distributed spatially on the nucleic acid backbone 100, 102, 104 and the respective attachment sites 118a, 118b of each attachment region 119 are densely packed, i.e. their spacing is preferably in the range of 1 nm to 50 nm. This allows amplification of a given target nucleic acid strand in a given attachment region 119 using chemistries further detailed in the description of FIG. 5.

Preferably, each attachment region 118, 119, in particular the respective attachment sites 122, 124, 124a, 124b, may hybridise specific predetermined target nucleic acids, such as a set of genes or genomic loci relevant to the diagnosis of a disease. This allows the capture of relevant sequence stretches of a liquid biopsy that is incubated with the backbones 100, 102, 104 of the sequencing scaffolds. Alternatively, attachment regions may be configured to hybridise target nucleic acids indirectly to the backbones 100, 102, 104. For example, target nucleic acids may be fragmented and then ligated to universal oligonucleotides (also termed adapters) that in turn hybridise with to the attachment regions 118, 119, in particular the respective attachment sites 122, 124, 124a, 124b.

In order to be able to determine the orientation of the backbones 100, 102, 104, the backbones 100, 102, 104 may optionally comprise a first orientation indicator 110, a second orientation indicator 112, and a third orientation indicator 114. The backbone 100 may further comprise a fourth orientation indicator 116.

The orientation indicators 110, 112, 114, 116 may be used to determine the orientation, directionality or polarity of the backbones 100, 102, 104. The orientation indicators 110, 112, 114, 116 may comprise at least one dye, in particular a fluorescent dye, such as fluorescein or a fluorescent protein. In addition, the dye of each of the orientation indicators 110, 112, 114, 116 may have different characteristics. The characteristics may include fluorescent emission characteristics, excitation characteristics or lifetime characteristics. This enables differentiating between the orientation indicators 110, 112, 114, 116 in an optical readout of the backbones 100, 102, 104, for example generated by a microscope, a cytometer, or an imaging cytometer. The orientation indicators 110, 112, 114, 116 are arranged spaced apart from each other. Preferably each orientation indicator 110, 112, 114, 116 is arranged at a particular end of the backbone 100, 102, 104. Thus, the orientation indicators 110, 112, 114, 116 enable differentiating between ends of the backbones 100, 102, 104. Ultimately, this enables determining the orientation, directionality or polarity of the backbones 100, 102, 104. The orientation indicators 110, 112, 114, 116 may be attached to a specific one of the attachment sites of the backbones 100, 102, 104 by a suitable oligonucleotide.

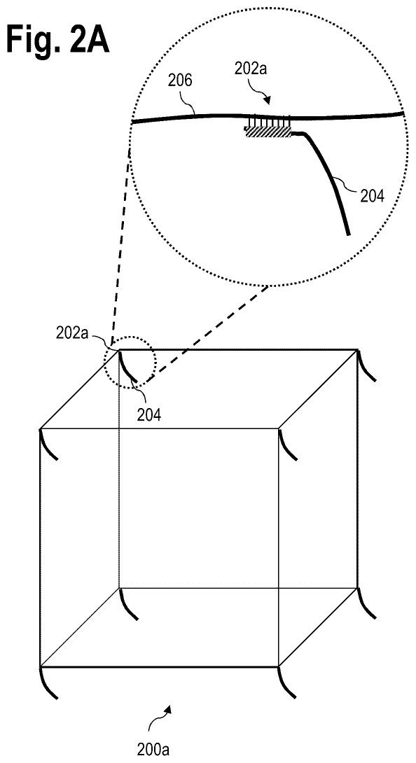

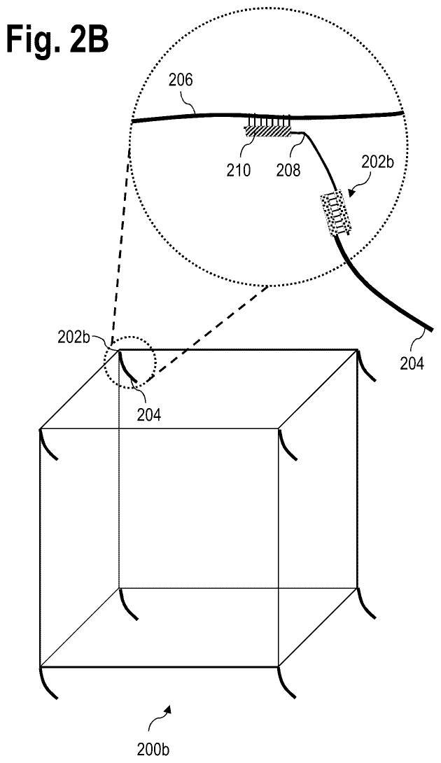

FIG. 2A and FIG. 2B are schematic views of nucleic acid backbones 200a, 200b of a sequencing scaffold. The backbones 200a, 200b have a cubic lattice structure, with attachment sites 202a, 202b in each corner of the backbones 200a, 200b. Target nucleic acid strands 204 may attach to the attachment sites 202a, 202b.

Specifically, FIG. 2A shows the attachment sites 202a that may be configured as integrated attachment sites, as described in connection with FIG. 1. A target nucleic acid strand 204 is attached directly to a nucleic acid strand 206 of the nucleic acid backbone 200a via the attachment site 202a. Each attachment site 202a also forms a respective attachment region.

Alternatively, the target nucleic acid strand 204 may be indirectly attached to the nucleic acid strand 206 of the nucleic acid backbone 200b. In this case, a docking strand 208 comprising the attachment site 202b is attached to the nucleic acid strand 206 and the target nucleic acid strand 204 is in turn attached to the docking strand 208 via the attachment site 202b. The attachment site 202b may be configured as a connected attachment site, as described in connection with FIG. 1. Each attachment site 202b also forms a respective attachment region.

There may be a plurality of docking strands 208 for each attachment region, each docking strand of the attachment region configured to hybridise the target nucleic acid strand 204. Specifically in this case of several docking strands 208, a plurality of attachment regions may then be arranged and distributed over the nucleic acid backbone 200b. Preferably, the attachment regions are spaced apart from each other in a range from 1 nm to 2000 nm, preferably in a range from 200 nm to 1000 nm. This enables a particularly dense arrangement of the target nucleic acid strands amplified in clonal groups on the attachment regions on the backbone. The docking strand 208 has an attachment oligonucleotide portion 210, that may be specifically complementary to a particular part of the nucleic acid strand 206.

The docking strand 208 may comprise a priming sequence, for initiating sequencing of the target nucleic acid 204 attached to the docking strand 208. The target nucleic acid strand 204 may be hybridised or ligated to the docking strand 208 prior to hybridising of the docking strand 208 with the target nucleic acid strand 204 being hybridised to the attachment site 202 of the backbone 200.

The attachment of the target nucleic acid strands 204 to either of the attachment sites 202a, 202b may be mediated by adapter oligonucleotides. The adapter oligonucleotides may be ligated to the target nucleic acid strands 204 before attachment of the target nucleic acid strands 204 to the backbone 200a, 200b and may be mediated by a complementarity of the attachment site or docking strand 208 to a part of the target nucleic acid (specific attachment). The latter principle may be used to pull a set of target nucleic acids from a mixture for example from a cell lysate or from a liquid biopsy onto the sequencing scaffold. In this sense the sequencing scaffold may be used to purify target nucleic acids by capture and subsequent washing before the sequencing of target nucleic acids is performed.

The adapter oligonucleotides may further comprise a universal priming sequence. This allows priming all target nucleic acid strands 204 in parallel for sequencing-by-synthesis.

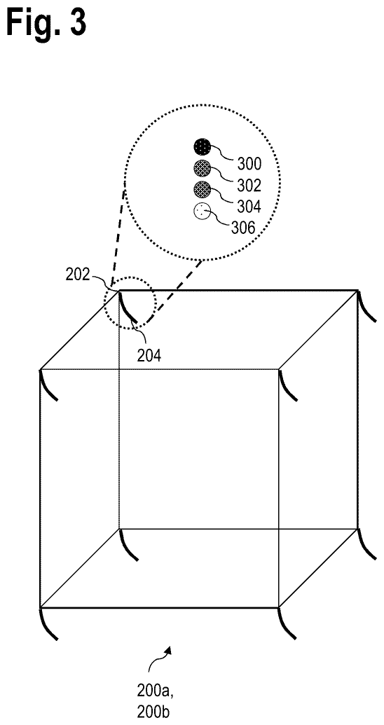

FIG. 3 is a schematic view of the nucleic acid backbone 200a, 200b with sequencing information of the attached target nucleic acid strand 204. The sequencing information is depicted as a temporal sequence or succession of individually read out optical signals 300, 302, 304, 306. The optical signal may be generated during sequencing by synthesis methods such as pyrosequencing, DNA nanoball sequencing, ILLUMINA sequencing, or other forms of sequencing-by-synthesis for example. Thus, the target nucleic acid strand acts as a template during synthesis of a complementary strand. For each nucleotide incorporated in the complementary strand, the optical signal may be specific to a particular nucleotide. For example, the optical signal 300 of the last incorporated nucleotide may be specific to incorporation of adenine, the optical signals 302, 304 may be specific to incorporation of thymine and the optical signal 306 may be specific to incorporation of guanine. Thus, by sequentially reading out the optical signals, the sequences of nucleic acids in the complementary strand may be determined, and therefore for the target nucleic acid strand. The sequential reading may be performed in a cyclical process that involve incorporation of a dye-labelled nucleotide, read-out of the incorporated dye, and dye removal or it may result from a continuous reading, which reads the incorporation of dye-labelled nucleotides at each attachment site or attachment region continuously.

Preferably, the docking strand 208 may be one of a first set of adapters, that all have the identical attachment oligonucleotide portion 210 or that have attachment oligonucleotide portions that bind to specific, predetermined first attachment sites 202 of the backbone 200. A second set of adapters may be provided with different attachment oligonucleotide portions, that bind to specific, predetermined second attachment sites. The target strands 204 may be from a first sample and are attached to the first attachment sites 202 via the first set of adapters and target strands from a second sample may be attached to the second attachment sites via the second set of adapters. The optical signals of each first and second attachment site may then be read out to determine sequences of the respective target strands. Since the target strands of the different samples are specifically attached to either the first or second attachment sites their respective sequences may be assigned to the respective samples. Thus, different samples may be sequenced at the same time whilst retaining the ability to assign each sequenced target strand to a particular one of the samples. This further increases sequencing throughput.

The optical signal may be detected by optically reading out the attachment sites, in particular the sequencing reaction at the attachment sites, for example, by means of an optical readout device, which may be a microscope such as a point-scanning confocal or a camera-based/widefield imaging system, for example a spinning disk microscope, a light sheet fluorescence microscope, a light field microscope, or a stereomicroscope. Further, the optical readout may be non-image-based readouts with at least one point detector or a line detector. The optical readout may be performed when the scaffold is in a flow-through device, such as a microfluidic chip.

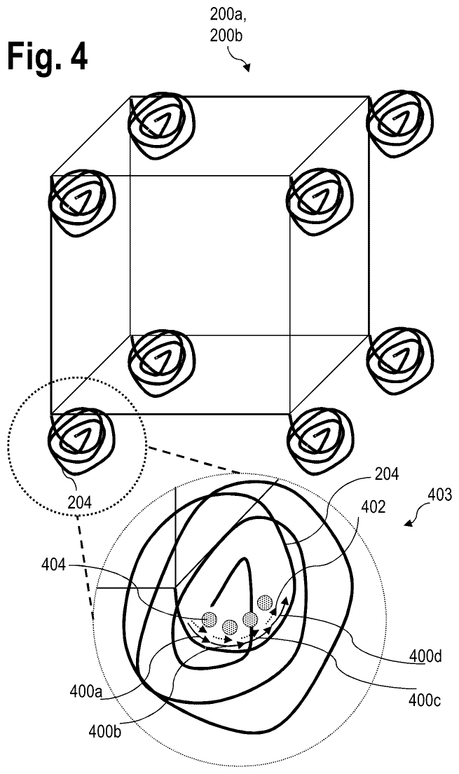

Prior to sequencing the target nucleic acid strands 204, the target strands 204 may be amplified while attached to the backbone 200a, 200b, for example by rolling circle amplification (RCA) as used in DNA nanoball sequencing, which is depicted schematically in FIG. 4. As shown in the schematic, a RCA reaction amplifies a target nucleic acid strand 204 attached to the backbone 200a, 200b in circles. This generates a concatenate of multiple copies 400a, 400b, 400c, 400d of the original target nucleic acid strand 204. A sequencing-by-synthesis reaction 402 can now be performed on said multiple copies 400a, 400b, 400c, 400d in parallel which leads to amplification of the optical signal 404 as each copy 400a, 400b, 400c, 400d generates the same signal 404 at the same time. The long concatenated strand consisting of multiple copies 400a, 400b, 400c, 400d tends to roll up into what is called a DNA nanoball 403, which can be readout by a microscope as a single spot.

Target nucleic acid strands 204 attached to the backbone 200a, 200b may further be amplified according to the chemistry and principle laid out in “Bentley, D., Balasubramanian, S., Swerdlow, H. et al. Accurate whole human genome sequencing using reversible terminator chemistry. Nature 456, 53-59 (2008). https://doi.org/10.1038/nature07517”. In this case multiple attachment sites of a first type and second type are needed, because this method uses a “pair of oligonucleotides in a forked adaptor configuration” to generate double-stranded blunt-ended nucleic acid fragments with a different i.e. a first and a second adaptor sequence on either end. Here we refer to this method as ILLUMINA sequencing. Embodiments of the present invention support this sequencing method as well as schematically shown in FIG. 5 by providing sequencing scaffolds with at least first attachment sites and second attachment sites (complementary to said first and said second adaptor sequence), wherein multiple first and second attachment sites are grouped into attachment regions on the nucleic acid backbone 200. Each region then holds several first and second attachment sites. For example 10-25, 25-50, 50-100, 100-1000 attachment sites to allow different levels of amplification. A spot of about 30 nucleic acid targets would generate a signal (˜30 fluorescence dye molecules per round) that can be well differentiated using for example a widefield, confocal, light sheet, super resolution or lightfield microscope as a readout and moderate excitation (laser) power in combination with typical pixel dwell/exposure times generating sufficient signal-to-noise and signal-to-background to perform robust base calling.

FIGS. 5A to 5J schematically show steps of a sequencing method.

In a step S500 of the method shown in FIG. 5A a target (double-stranded) nucleic acid strand 500 is introduced. These strands 500 may be obtained via several fragmentation methods from genomic DNA that derives from a biological sample (e.g. cell lysate, tissue biopsy, liquid biopsy, water sample). In a step S502 adapter oligonucleotides 502a of a first type and adapter oligonucleotides 502b of a second type are ligated to the target nucleic acid strand 500. A “pair of oligonucleotides in a forked adaptor configuration” as described in Bentley et al. 2008 may be used in this step to ensure that either end of the target nucleic acid strand 500 is connected to a different adapter. This first adapter 502a is symbolized by a dotted line attached to the target nucleic acid 500, the second adapter 502b is symbolized by a dashed line. The first adapter 502a (dotted line) is at least partially complementary to the first attachment site 124a (dotted line) and the second adapter 502b (dashed line) is at least partially complementary to the second attachment site 124b (dashed line). Such that in a step S504 the (double-stranded) nucleic acid-adapter conjugate is melted to generate single-stranded target nucleic acid-adaptor conjugate 504, which is in turn hybridized to the complementary first attachment site 124a (dotted line) of the attachment region 119.

In a step S506 shown in FIG. 5B a complementary strand 504b is synthesized, wherein synthesis is primed by universal priming sequences present on the attachment sites 124a, 124b. This effectively connects the target nucleic strand to the attachment site 124a of the attachment region 119.

In a step S508 shown in FIG. 5C one of the double strands of the is melted allowing the release of the unbound target nucleic acid fragment-adaptor conjugate 504.

Through a cyclical process including steps S510-S522 schematically shown in FIGS. 5D to 5J the physically linked target nucleic acid-adaptor-conjugate 504b is now amplified on the attachment region, which generates a densely packed spot of multiple copies of target nucleic acid-adaptor-conjugates, which can be sequenced in parallel using sequencing-by-synthesis approaches, as described above.

FIG. 5D shows a step S510 in which the second adaptor 502b is hybridised to a neighbouring attachment site 124b. FIG. 5E shows step S512 DNA synthesis of a complementary strand 504a (cycle n). FIG. 5F shows step S514 melting of the complementary strands 504a, 504b (cycle n). FIG. 5G shows a step S516 with reannealing/rehybridization of the complementary strands 504a, 504b (cycle n). FIG. 5H shows step S518 with further DNA synthesis of further complementary strands 504c, 504d (cycle n+1). FIG. 5I shows step S520 with melting of further complementary strands 504c, 504d (cycle n+1). FIG. 5J shows step S522 with sequencing following to cycle n+m (amplificiation of steps S510 to S518 may proceed until all attachment sites 124a, 124b per attachment region 119 are occupied or stopped before at a sufficient level of amplification).

Sequencing may then be performed by synthesis or by another means such as for example by hybridization or by ligation.

While amplification may be desirable in most cases, there are cases in which a simpler non-amplification chemistry is preferred to enable not only sequencing of target nucleic acid fragments, but also the enumeration of the same. Like for example in diagnostic applications to a level of contamination of water with a pathogen may be determined in this way, or in the case of cancer copy number variation may be assessed.

A sequencing without amplification using bright dyes and particularly sensitive microscopic detection platforms, which have suitable high NA, high transmission optics, and sensitive detectors such as back-thinned, back-illuminated CMOS sensors is equally possible.

Identical or similarly acting elements are designated with the same reference signs in all Figures. As used herein the term “and/or” includes any and all combinations of one or more of the associated listed items and may be abbreviated as “/”. Individual features of the embodiments and all combinations of individual features of the embodiments among each other as well as in combination with individual features or feature groups of the preceding description and/or claims are considered disclosed.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

LIST OF REFERENCE SIGNS

| | |
|---|---|
| 100, 102, 104, 200a, 200b | Nucleic acid backbone |
| 110, 112, 114, 116 | Orientation indicators |
| 118, 119 | Attachment region |
| 122, 202a | Integrated attachment site |
| 124, 124a, 124b, 202b | Connected attachment site |
| 204 | Target nucleic acid strand |
| 206 | Nucleic acid strand of backbone |
| 208 | Docking strand |
| 210 | Attachment oligonucleotide portion |
| 300, 302, 304, 306, 404 | Optical signal |
| 400a, b, c, d | Rolling circle amplified target nucleid |
| | acid strand |
| 402 | Sequencing-by-synthesis |
| 403 | DNA Nanoball |
| 500 | Double-stranded target nucleic acid |
| | fragment-adaptor conjugate |
| 502a, 502b | Adapter |
| 504, 504a, 504b, 504c, | Single-stranded target nucleic acid- |
| 504d | adaptor conjugate |

1: A sequencing scaffold for attachment of target nucleic acid strands, the sequencing scaffold comprising,
at least one nucleic acid backbone with a plurality of attachment regions at predetermined positions, each attachment region comprising at least one attachment site,
wherein the attachment sites are configured to hybridise the target nucleic acid strands, and
wherein the nucleic acid backbone has a three-dimensional lattice structure, and the attachment regions are arranged on the lattice structure of the nucleic acid backbone in three dimensions.

2: The sequencing scaffold according to claim 1, further comprising at least a first orientation indicator, a second orientation indicator, and a third orientation indicators.

3: The sequencing scaffold according to claim 2, wherein the first orientation indicators, the second orientation indicator, and the third orientation indicator are spaced apart from each other, or arranged on opposite ends of the nucleic acid backbone.

4: The sequencing scaffold according to claim 1, wherein the attachment regions are arranged on the nucleic acid backbone in several planes, and the planes, are arranged at a distance from each other in a direction perpendicular to the planes.

5: The sequencing scaffold according to claim 4, wherein the distance between the planes is in a range from 5 nm to 6400 nm.

6: The sequencing scaffold according to claim 1, wherein a largest spatial extent of the nucleic acid backbone is in a range of 10 nm to 10000 nm.

7: The sequencing scaffold according to claim 1, wherein the attachment regions are spaced apart from each other in a range from 1 nm to 5000 nm.

8: The sequencing scaffold according to claim 1, wherein the nucleic acid backbone comprises scaffold strands and staple strands, the staple strands being configured to bind to the scaffold strands at predetermined positions to fold the scaffold strands into a predetermined shape.

9: The sequencing scaffold according to claim 8, wherein at least some of the staple strands comprise the attachment sites.

10: The sequencing scaffold according to claim 1, wherein the nucleic acid backbone comprises double stranded nucleic acid strands, and the attachment sites comprise single stranded oligonucleotides.

11: The sequencing scaffold according to claim 1, wherein each attachment site comprises a unique oligonucleotide sequence.

12: The sequencing scaffold according to claim 1, wherein each attachment region comprises at least a first set of attachment sites configured to hybridise a first adapter oligonucleotide, and a second set of attachment sites configured to hybridise a second adapter oligonucleotide.

13: The sequencing scaffold according to claim 12, wherein the first adapter oligonucleotide and the second adapter oligonucleotide are configured to be ligated to the target nucleic acid strands.

14: The sequencing scaffold according to claim 1, further comprising a second nucleic acid backbone, wherein the first nucleic acid backbones and the second nucleic acid backbone are linked to each other.

15: A method for sequencing target nucleic acid strands, the method comprising:
providing a sequencing scaffold comprising at least one nucleic acid backbone according to claim 1,
attaching the target nucleic acid strands to attachment sites of the at least one nucleic acid backbone of the sequencing scaffold,
sequencing the target nucleic acid strands according to a sequencing method, the sequencing method comprising at least amplifying the attached target nucleic acid strands and iteratively optically reading out the attachment sites.

16: The method according to claim 15, wherein prior to the attaching the target nucleic acid strands to the attachment sites, the first adapter oligonucleotide and the second adapter oligonucleotide are ligated to each target nucleic acid strand.
